(19) United States
(12) Patent Application Publication
(10) Pub. No.: US 2007/0232609 A1
(43) Pub. Date: Oct. 4, 2007

(54) Novel Antiparasitic Combination of Active Compounds

(75) Inventor: Gisela Greif, Remagen (DE)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: BAYER HEALTHCARE AG, LEVERKUSEN (DE)

(21) Appl. No.: 11/574,614

(22) PCT Filed: Mar. 9, 2006
(86) PCT No.: PCT/EP05/09084

§ 371 Date: Mar. 2, 2007

(30) Foreign Application Priority Data
Sep. 2, 2004 (DE) 10 2004 042 958.8

(51) Int. Cl. A61K 31/4184; A61K 31/53

(52) U.S. Cl. 514/242; 514/394

(57) ABSTRACT

The present invention relates to the combined use of substituted benzimidazoles and 1,2,4-triazine compounds against parasitic protozoa, in particular coccidia.

The present invention relates to the combined use of substituted benzimidazoles and 1,2,4-triazine compounds against parasitic protozoa, in particular coccidia.

Substituted benzimidazoles and their use as insecticides, fungicides and herbicides are already known (EP-A 87 375, 152 360, 181 826, 239 508, 260 744, 266 984, U.S. Pat. Nos. 3,418,318, 3,472,865, 3,576,818, 3,728,994). Halogenated benzimidazoles and their action as anthelmintics, coccidiostatics and pesticides are known (DE-A 2 047 369, EP 597 304 A1). The substituted benzimidazoles which are preferably used in accordance with the present invention are described in WO 00/04022 and WO 00/68225.

Mixtures of nitro-substituted benzimidazoles and polyether antibiotics have been disclosed as compositions against coccidiosis (U.S. Pat. No. 5,331,003). Mixtures of substituted benzimidazoles with polyether antibiotics or synthetic agents against coccidiosis are known from WO 96/38140 as compositions for controlling parasitic protozoa.

The combination of substituted benzimidazoles with 1,2,4-triazines, which combination is highly suitable for controlling parasitic protozoa, has hitherto not been described.

Coccidiosis may be mentioned as an important example of a disease caused by single-cell parasites (protozoa). In particular, in poultry breeding, it can cause great losses. To avoid these, the stocks are treated prophylactically with agents against coccidiosis. Development of resistance against the agents used causes serious problems even shortly after the introduction of the agents. On the other hand, by using chemically entirely novel agents against coccidiosis, in particular combinations, it is possible to control even polyresistant parasite strains.

Accordingly, the invention relates to:

Products comprising at least one substituted benzimidazole effective against parasitic protozoa and at least one 1,2,4-triazine derivative.

Preferred benzimidazoles are those of the formula (I) 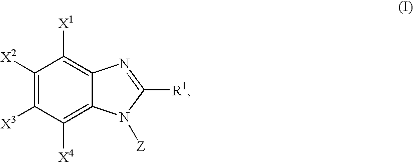
in which
Z represents hydrogen or the radical —CHR²R³,
R¹ represents fluoroalkyl,
R² represents hydrogen or alkyl,
R³ represents a radical of the formula 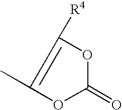
   or represents a radical of the formula 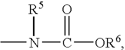
R⁴ represents alkyl,
R⁵ represents alkyl or substituted phenyl,
R⁶ represents alkyl,
X¹, X², X³ and X⁴ independently of one another represent hydrogen, halogen, halo-alkyl, haloalkoxy, haloalkylthio or haloalkylsulphonyl,
   or else
X² and X³ or X³ and X⁴ together represent a dioxyhaloalkylene radical.

The formula (I) provides a general definition of the substituted benzimidazoles according to the invention.
R¹ preferably represents C₁-C₄-fluoroalkyl,
R² preferably represents hydrogen or C₁-C₄-alkyl,
R⁴ preferably represents C₁-C₄-alkyl,
R⁵ preferably represents C₁-C₆-alkyl or phenyl which is optionally mono- or polysubstituted by C₁-C₄-alkyl, C₁-C₄-haloalkyl, halogen, nitro, C₁-C₄-alkoxy, C₁-C₄-haloalkoxy or methylene- or ethylenedioxy which is optionally mono- or poly-substituted by halogen,
R⁶ preferably represents C₁-C₄-alkyl,
X¹, X², X³ and X⁴ independently of one another preferably represent hydrogen, F, Cl, Br, C₁-C₄-haloalkyl, C₁-C₄-haloalkoxy, C₁-C₄-haloalkylthio, C₁-C₄-halo-alkylsulphonyl, or
X² and X³ or X³ and X⁴ according to a further preferred embodiment together represent a dioxyhalo-C₁-C₄-alkylene radical.
R¹ particularly preferably represents CF₃, CHF₂ or CHF.
R² particularly preferably represents hydrogen, methyl, ethyl, n-propyl or iso-propyl.
R⁴ particularly preferably represents methyl, ethyl, n-propyl or isopropyl.
R⁵ particularly preferably represents C₁-C₆-alkyl.
R⁶ particularly preferably represents methyl or ethyl.
X¹, X², X³ and X⁴ particularly preferably independently of one another represent hydrogen, F, Cl, Br, CF₃, CHF₂, CH₂F, OCF₃, OCH₂F, OCHF₂, SCF₃, SCHF₂, SCH₂F, SO₂CF₃, SO₂CHF₂, SO₂CH₂F.
X² and X³ or X³ and X⁴ according to a further embodiment together also particularly preferably represent a radical —O—CF₂—O—, —O—CF₂—CF₂—O—, —O—CF₂—CF₂—CF₂—O—, —O—CF₂—CHF—O—, —O—CClF—CClF—O—, —O—CHF—O—, —O—CHF—CHF—O— or —O—CClF—O—.

According to a very particularly preferred embodiment, R³ represents a radical of the formula

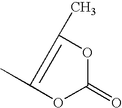

According to a further very particularly preferred embodiment, R³ represents a radical of the formula 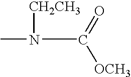
R¹ very particularly preferably represents —CF₃.
R² very particularly preferably represents hydrogen.
R⁴ very particularly preferably represents methyl.
X¹ very particularly preferably represents Cl or Br.
X² very particularly preferably represents hydrogen.
   and
X³ and X⁴ very particularly preferably together represent —OCF₂—CF₂—O—.

Alkyl denotes a straight-chain or branched hydrocarbon radical having 1 to 8, preferably 1 to 6, particularly preferably 1 to 4, carbon atoms, such as, for example, methyl, ethyl, propyl, isopropyl, n-butyl, sec-butyl, tert-butyl.

Alkylene denotes a straight-chain or branched hydrocarbon radical having 1 to 4, preferably 1 to 3, particularly preferably 1 or 2, carbon atoms, which radical is attached via two different positions.

Haloalkyl denotes an alkyl radical as defined above in which one or more, in particular 1 to 3, hydrogen atoms have been replaced by a halogen atom, in particular by fluorine, chlorine or bromine.

Correspondingly, fluoroalkyl radical denotes an alkyl radical in which 1 to all hydrogen atoms have been replaced by fluorine atoms; preference is given to perfluoroalkyl radicals, for example trifluoromethyl or pentafluoroethyl.

Haloalkoxy denotes a straight-chain or branched alkoxy radical having 1 to 8, preferably 1 to 6, particularly preferably 1 to 4, carbon atoms, in which radical one or more, in particular 1 to 3, hydrogen atoms have been replaced by a halogen atom, in particular by fluorine, chlorine or bromine; for example —OCF₃.

Haloalkylthio denotes a straight-chain or branched alkylthio radical having 1 to 8, preferably 1 to 6, particularly preferably 1 to 4, carbon atoms, in which radical one or more, in particular 1 to 3, hydrogen atoms have been replaced by a halogen atom, in particular by fluorine, chlorine or bromine; for example CF₃S—.

Haloalkylsulphonyl denotes a straight-chain or branched alkylsulphonyl radical having 1 to 8, preferably 1 to 6, particularly preferably 1 to 4, carbon atoms, in whose alkyl moiety one or more, in particular 1 to 3, hydrogen atoms have been replaced by a halogen atom, in particular by fluorine, chlorine or bromine.

According to one embodiment, Z in formula (I) represents hydrogen and the other substituents can have the meanings given above, including the preferred and particularly preferred meanings. The compound of the formula (I-A) (see WO 00/04022) may be mentioned as a preferred example of this embodiment:

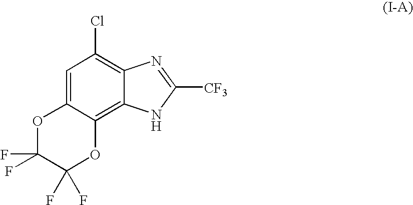

According to a preferred further embodiment, Z in formula (I) represents the radical —CHR²R³ and the other substituents may have the meanings given above, including the preferred and particularly preferred meanings. The compound of the formula (I-B) (see WO 00/04022) and in particular the compound of the formula (I-C) (see WO 00/68225) may be mentioned as preferred examples of this embodiment:

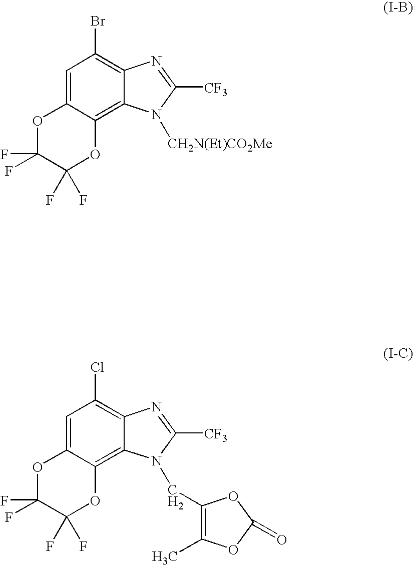

Hitherto, compounds of the formula (I) in which Z represents hydrogen have been known as intermediates for the preparation of effective benzimidazole active compounds. Surprisingly, it has now been found that the compounds of the formula (I) in which Z represents hydrogen for their part are highly effective against parasitic protozoa (as illustrated in more detail below). Therefore, according to a further aspect, the present invention relates to the use of compounds of the formula (I) in which Z represents hydrogen for controlling parasitic protozoa, in particular in animal husbandry and animal breeding. Preferred and particularly preferred compounds of the formula (I) in which Z represents hydrogen are those in which the other substituents have the meanings given above as being preferred and particularly preferred. For an especially preferred example, reference may be made to the compound of the formula (I-A). The preparation of such compounds is known or can be carried out analogously to known methods, see, for example, WO 00/04022, WO 00/68225 and EP 597 304 A1, and the literature cited therein.

1,2,4-Triazines which are active against parasitic protozoa are known. Preferred 1,2,4-triazines are represented by the formula (II): 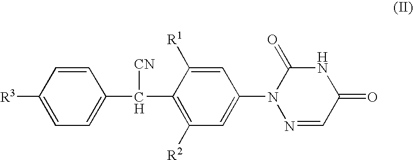
in which
R¹ and R² independently of one another represent hydrogen or Cl and
R³ represents fluorine or chlorine.

Particularly preferred examples are:

Clazuril (R¹=Cl, R²=H, R=Cl in formula (II))

Letrazuril (R¹=C¹, R²=C¹, R³=F in formula (II)) and

Diclazuril (R¹=Cl, R²=C¹, R³=Cl in formula (II)).

From among these 1,2,4-triazines, diclazuril is most preferred.

Depending on the nature and number of substituents, the active compounds mentioned above may, if appropriate, be present as geometrical and/or optical isomers or regioisomers or isomer mixtures thereof of varying composition. According to the invention, it is possible to use both the pure isomers and the isomer mixtures.

If the active compounds are capable of forming salts, the application in the form of pharmaceutically acceptable salts is also possible.

Furthermore suitable is, if appropriate, also the use of hydrates or other solvates of the active compounds or their salts.

The active compounds have favourable toxicity to warm-blooded animals and are suitable for the control of parasitic protozoa which occur in animal husbandry and animal breeding in the case of useful, breeding, zoo, laboratory and experimental animals and pets. At the same time, they are active against all or individual stages of development of the pests and also against resistant and normally sensitive strains. By means of the control of the parasitic protozoa, illness, cases of death and yield reductions (e.g. in the production of meat, milk, wool, hides, eggs, honey etc.) should be decreased, so that simpler and more economical animal husbandry is possible due to the use of the active compounds.

The parasitic protozoa include:

Mastigophora (Flagellata) such as, for example, Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example, Giardia lamblia, G. canis.

Sarcomastigophora (Rhizopoda) such as Entamoebidae, for example, Entamoeba histolytica, Hartmanellidae, for example, Acanthamoeba sp., Hartmanella sp.

Apicomplexa (Sporozoa) such as Eimeridae, for example, Eimeria acervulina, E. adenoides, E. alabahmensis, E. anatis, E. anseris, E. arloingi, E. ashata, E. aubumensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Hammon dia heyderni, Isospora belli, I. canis, I. felis, I. ohioensis, I. rivolta, I. spec., I. suis, Neospora spec., Neospora carinum, Neospora hugesi, Neospora caninum, Cystisospora spec., Cryptosporidium spec. such as Toxoplasmadidae, for example, Toxoplasma gondii, such as Sarcocystidae, for example, Sarcocystis bovicanis, S. bovihominis, S. neurona, S. ovicanis, S. ovifelis, S. spec., S. suihominis such as Leucozoidae, for example, Leucozytozoon simondi, such as Plasmodiidae, for example, Plasmodium berghei, P. falciparum, P. malariae, P. ovale, P. vivax, P. spec., such as Piroplasmea, for example, Babesia argentina, B. bovis, B. canis, B. spec., Theileria parva, Theileria spec., such as Adeleina, for example, Hepatozoon canis, H. spec.

Furthermore Myxospora and Microspora, for example, Glugea spec. Nosema spec.

Furthermore Pneumocystis carinii, and also Ciliophora (Ciliata) such as, for example, Balantidium coli, Ichthiophthirius spec., Trichodina spec., Epistylis spec.

The active compounds and active compound combinations according to the invention are also active against protozoa which occur as parasites in insects. Those which may be mentioned are parasites of the strain Microsporida, in particular of the genus Nosema. Particular mention may be made of Nosema apis in the case of the honeybee.

The useful and breeding animals include mammals, such as, for example, cattle, horses, sheep, pigs, goats, camels, water buffalo, donkeys, rabbits, fallow deer, reindeer, fur-bearing animals such as, for example, mink, chinchilla, racoons, birds, such as, for example, hens, geese, turkeys, ducks, doves, bird species for keeping at home and in zoos. Useful and ornamental fish are furthermore included.

The laboratory and experimental animals include mice, rats, guinea pigs, golden hamsters, dogs and cats.

The pets include dogs and cats.

The fish include useful, breeding, aquarium and ornamental fish of all age levels, which live in fresh and salt water. The useful and ornamental fish include, for example, carp, eels, trout, whitefish, salmon, bream, roach, rudd, chub, sole, plaice, halibut, Japanese yellowtail (Seriola quinqueradiata), Japanese eel (Anguilla japonica), red sea bream (Pagurus major), sea bass (Dicentrarchus labrax), grey mullet (Mugilus cephalus), pompano, gilthead sea bream (Sparus auratus), Tilapia ssp., Chichlidae species such as, for example, Plagioscion, Channel catfish. The compositions according to the invention are particularly suitable for the treatment of fry, e.g. carp of 2 to 4 cm body length. The compositions are also very highly suitable in eel feeding.

Administration can be carried out both prophylactically and therapeutically.

The administration of the active compounds is carried out directly or enterally, parenterally, dermally or nasally in the form of suitable preparations.

Enteral administration of the active compounds takes place, for example, orally in the form of powders, suppositories, tablets, capsules, pastes, drinks, granules, drenches, boli, medicated feed or drinking water. Dermal administration takes place, for example, in the form of dipping, spraying, bathing, washing, pouring on and spotting on, and dusting. Parenteral administration takes place, for example, in the form of injection (intramuscular, subcutaneous, intravenous, intraperitoneal) or by means of implants.

Suitable preparations are:

Solutions such as injection solutions, oral solutions, concentrates for oral administration after dilution, solutions for use on the skin or in body cavities, pour-on formulations, gels;

emulsions and suspensions for oral or dermal administration and for injection; semi-solid preparations;

formulations in which the active compound is incorporated in an ointment base or in an oil-in-water or water-in-oil emulsion base.

Solid preparations such as powders, premixes or concentrates, granules, pellets, tablets, boli, capsules; aerosols and inhalations, active compound-containing shaped articles.

Injection solutions are administered intravenously, intramuscularly and subcutaneously.

Injection solutions are prepared by dissolving the active compound in a suitable solvent and possibly adding additives such as solubilizers, acids, bases, buffer salts, antioxidants, preservatives. The solutions are sterile-filtered and filled into containers.

Solvents which may be mentioned are: physiologically tolerable solvents such as water, alcohols such as ethanol, butanol, benzyl alcohol, glycerol, hydrocarbons, propylene glycol, polyethylene glycols, N-methylpyrrolidone, and mixtures thereof.

If appropriate, the active compounds can also be dissolved in physiologically tolerable vegetable or synthetic oils which are suitable for injection.

Solubilizers which may be mentioned are: solvents which promote the dissolution of the active compound in the main solvent or prevent its precipitation. Examples are polyvinylpyrrolidone, polyethoxylated castor oil, polyethoxylated sorbitan ester.

Preservatives are: benzyl alcohol, trichlorobutanol, esters of p-hydroxybenzoic acid, n-butanol.

Oral solutions are administered directly. Concentrates are used orally after prior dilution to the use concentration. Oral solutions and concentrates are prepared as described above in connection with the injection solutions, it being possible to dispense with sterile operation.

Solutions for use on the skin are spotted on, painted on, rubbed in, squirted or sprayed on or applied by dipping, bathing or washing. These solutions are prepared as described above in connection with the injection solutions.

It may be advantageous to add thickeners during preparation. Thickeners are: inorganic thickeners such as bentonites, colloidal silica, aluminium monostearate, organic thickeners such as cellulose derivatives, polyvinyl alcohols and their copolymers, acrylates and metacrylates.

Gels are applied to or painted onto the skin or introduced into body cavities. Gels are prepared by mixing solutions, which have been prepared as described in connection with the injection solutions, with sufficient thickener to form a clear composition with an ointment-like consistency. Thickeners employed are the thickeners indicated further above.

Pour-on formulations are poured or squirted onto limited areas of the skin, the active compound either penetrating the skin and acting systemically or being dispersed on the surface of the body.

Pour-on formulations are prepared by dissolving, suspending or emulsifying the active compound in suitable skin-tolerable solvents or solvent mixtures. If appropriate, further auxiliaries such as colorants, absorption-promoting substances, antioxidants, sunscreen agents and/or adherents are added.

Solvents which may be mentioned are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol monobutyl ether, ketones such as acetone, methyl ethyl ketone, aromatic and/or aliphatic hydrocarbons, vegetable or synthetic oils, DMF, dimethylacetamide, N-methylpyrrolidone, 2-dimethyl-4-oxymethylene-1,3-dioxolane.

Colorants are all colorants approved for use on animals and which can be dissolved or suspended.

Absorption-promoting substances are, for example, DMSO, spreading oils such as isopropyl myristate, dipropylene glycol pelargonate, silicone oils, esters of fatty acids, triglycerides, fatty alcohols.

Antioxidants are sulphites or metabisulphites as potassium metabisulphite, ascorbic acid, butylhydroxytoluene, butylhydroxyanisole, tocopherol.

Sunscreen agents are, for example, substances from the benzophenones or novantisolic acid class.

Adherents are, for example, cellulose derivatives, starch derivatives, polyacrylates, natural polymers such as alginates, gelatine.

Emulsions can be used orally, dermally or as injections.

Emulsions are either of the water-in-oil type or of the oil-in-water type.

They are prepared by dissolving the active compounds either in the hydrophobic or in the hydrophilic phase and homogenizing this with the solvent of the other phase with the aid of suitable emulsifiers and, if appropriate, further auxiliaries such as colorants, absorption-promoting substances, preservatives, antioxidants, sunscreen agents and/or viscosity-increasing substances.

Hydrophobic phases (oils) which may be mentioned are: paraffin oils, silicone oils, natural vegetable oils such as sesame oil, almond oil, castor oil, synthetic triglycerides such as caprylic/capric acid biglyceride, triglyceride mixture with vegetable fatty acids of chain length C₈₋₁₂ or other specially selected natural fatty acids, partial glyceride mixtures of saturated or unsaturated fatty acids possibly also containing hydroxyl groups, mono- and diglycerides of the C₈/C₁₀ fatty acids.

Esters of fatty acids such as ethyl stearate, di-n-butyryl adipate, hexyl laurate, dipropylene glycol pelargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleates, decyl oleates, ethyl oleate, ethyl lactates, waxy fatty acid esters such as dibutyl phthalate, diisopropyl adipate, ester mixtures related to the latter, inter alia fatty alcohols such as isotridecyl alcohol, 2-octyldodecanol, cetylstearyl alcohol, oleyl alcohol.

Fatty acids such as, for example, oleic acid and its mixtures.

Hydrophilic phases which may be mentioned are: water, alcohols such as, for example, propylene glycol, glycerol, sorbitol and their mixtures.

Emulsifiers which may be mentioned are:

nonionic surfactants, e.g. polyethoxylated castor oil, polyethoxylated sorbitan monooleate, sorbitan monostearate, glycerol monostearate, polyoxyethyl stearate, alkylphenol polyglycol ethers;

ampholytic surfactants such as di-Na N-lauryl-β-iminodipropionate or lecithin;

anionic surfactants, such as Na laurylsulphate, fatty alcohol ether sulphates, mono/dialkyl polyglycol ether orthophosphate monoethanolamine salt;

cationic surfactants such as cetyltrimethylammonium chloride.

Further auxiliaries which may be mentioned are:

viscosity-increasing and emulsion-stabilizing substances such as carboxymethyl-cellulose, methylcellulose and other cellulose and starch derivatives, polyacrylates, alginates, gelatine, gum arabic, polyvinylpyrrolidone, polyvinyl alcohol, copolymers of methyl vinyl ether and maleic anhydride, polyethylene glycols, waxes, colloidal silica or mixtures of the substances mentioned.

Suspensions can be used orally, dermally or as an injection. They are prepared by suspending the active compound in a suspending agent, if appropriate with addition of further auxiliaries such as wetting agents, colorants, absorption-promoting substances, preservatives, antioxidants, sunscreen agents.

Suspending agents which may be mentioned are all homogeneous solvents and solvent mixtures.

Wetting agents (dispersing agents) which may be mentioned are the surfactants indicated further above.

Further auxiliaries which may be mentioned are those indicated further above.

Semisolid preparations can be administered orally or dermally. They differ from the suspensions and emulsions described above only by their higher viscosity.

To prepare solid preparations, the active compounds are mixed with suitable supports, if appropriate with addition of auxiliaries, and brought into the desired form.

Supports which may be mentioned are all physiologically tolerable solid inert substances. Those which are used are inorganic and organic substances. Inorganic substances are, for example, sodium chloride, carbonates such as calcium carbonate, hydrogen carbonates, aluminium oxides, silicas, argillaceous earths, precipitated or colloidal silica, phosphates.

Organic substances are, for example, sugar, cellulose, foodstuffs and feedstuffs such as powdered milk, animal meals, cereal meals and shreds, starches.

Auxiliaries are preservatives, antioxidants and colorants which have already been mentioned further above.

Further suitable auxiliaries are lubricants and glidants such as, for example, magnesium stearate, stearic acid, talc, bentonites, disintegration-promoting substances such as starch or crosslinked polyvinylpyrrolidone, binding agents such as, for example, starch, gelatine or linear polyvinylpyrrolidone and also dry binding agents such as microcrystalline cellulose.

The active compounds can be present in combination with synergists or with other active compounds.

Suitable other active compounds are in particular polyether antibiotics, such as, for example:

amprolium, in some cases in combination with folic acid antagonists

robenidine

monensin

salinomycin

lasalocid

narasin

semduramicin and

in particular maduramicin.

Ready-to-use preparations contain the active compounds in each case in concentrations of from 0.005 ppm to 50 ppm, preferably from 0.1 to 10 ppm.

In general, it has proved advantageous to administer amounts from approximately 0.05 to approximately 50 mg, preferably 0.1 to 20 mg, of active compound per kg of body weight per day to achieve effective results.

In the mixture with other agents against coccidiosis or polyether antibiotics, the active compounds according to the invention are in the ratio 1 to 0.01-50 to 1 to 1-50. The ratio 1 to 25 is preferred.

The active compounds can also be administered to the animals together with the feed or drinking water.

Feedstuffs and foodstuffs contain 0.005 to 250 ppm, preferably 0.05 to 100 ppm, of the active compound in combination with a suitable edible material.

Such a feedstuff and foodstuff can be used both for curative purposes and for prophylactic purposes.

Such a feedstuff or foodstuff is prepared by mixing a concentrate or a premix which contains 0.5 to 30%, preferably 1 to 20%, by weight of an active compound as a mixture with an edible organic or inorganic carrier with customary feedstuffs. Edible carriers are, for example, maize flour or maize and soya bean flour or mineral salts, which preferably contain a small amount of an edible dust prevention oil, e.g. maize oil or soya oil. The premix obtained in this way can then be added to the complete feedstuff before feeding it to the animals.

By way of example, use in coccidiosis may be mentioned:

For the healing and prophylaxis, for example, of coccidiosis in poultry, in particular in hens, ducks, geese and turkeys, 0.005 to 100 ppm, preferably 0.05 to 100 ppm, of an active compound are mixed with a suitable edible material, e.g. a nutritious feedstuff. If desired, these amounts can be increased, particularly if the active compound is well tolerated by the recipient. Correspondingly, administration can be carried out via the drinking water.

For the treatment of individual animals, e.g. in the case of the treatment of coccidiosis in mammals or of toxoplasmosis, amounts of active compound of 0.05 to 100 mg/kg of body weight are preferably administered daily in order to achieve the desired results. In spite of this, it may occasionally be necessary to depart from the amounts mentioned, in particular depending on the body weight of the experimental animal or on the type of administration method, but also because of the animal genus and its individual reaction to the active compound or the nature of the formulation and the time or the interval at which it is administered. Thus in certain cases it may be sufficient to manage with less than the abovementioned minimum amount, while in other cases the upper limit mentioned must be exceeded. When administering relatively large amounts, it may be advisable to divide these into several individual administrations during the course of the day.

The efficacy of the compounds according to the invention can be confirmed, for example, in cage experiments with the following experimental arrangement, in which the animals are treated with the respective individual components and with the mixtures of the individual components.

An active compound-containing feed is prepared such that the required amount of active compound is basically mixed with a nutritionally balanced animal feed, e.g. with the chick feed indicated below.

If a concentrate or a premix is to be prepared, which is finally to be diluted in the feed to the values mentioned in the experiment, in general approximately 1 to 30%, preferably approximately 10 to 20%, by weight of active compound are mixed with an edible organic or inorganic carrier, e.g. maize and soya meal or mineral salts which contain a small amount of an edible dedusting oil, e.g. maize oil or soya bean oil. The premix thus obtained can then be added to the complete poultry feed before administration.

A suitable example of the use of the substances according to the invention in the poultry feed is the following composition.

| | |
|---|---|
| 52.00% | of feed cereal shreds, that is: 40% maize, 12% wheat |
| 17.00% | of soya shreds extr. |
| 5.00% | of maize gluten feed |
| 5.00% | of wheat feed meal |
| 3.00% | of fish meal |
| 3.00% | of mineral mixture |
| 3.00% | of alfalfa meal |
| 2.50% | of vitamin premix |
| 2.00% | of wheat germs, comminuted |
| 2.00% | of soya oil |
| 2.00% | of meat and bone meal |
| 1.50% | of whey powder |
| 1.00% | of molasses |
| 1.00% | of brewer's yeast, bound to brewer's grains |
| 100.00% | |

Such a feed contains 18% raw protein, 5% raw fibre, 1% Ca, 0.7% P and, per kg, 1200 I.U. of vitamin A, 1200 I.U. of vitamin D3, 10 mg of vitamin E, 20 mg of zinc bacitracin.

Cage Experiment on Coccidiosis/Chicks

8- to 12-day-old male chicks (e.g. LSL Brinkschulte/Senden) which have been reared coccidia-free receive the compounds according to the invention (test substances) in the concentration indicated in ppm with the feed from 3 days before (day −3) infection (=a.i.) until 8 (9) days after infection (=p.i.). 3 animals are kept in each cage. One or more groups of this type are employed per dose. Infection is carried out by means of a stomach tube directly into the crop with approximately 100 000 sporulated oocysts of Eimeria acervulina and with approximately 30 000 oocysts each of E. maxima and 40 000 sporulated oocysts of E. tenella. These are highly virulent strains. The exact infection dose is adjusted so that, if possible, one of three experimentally infected untreated chicks dies due to the infection. For assessment of the efficacy, the following criteria are taken into account: weight increase from the start of the experiment to the end of the experiment, death rate due to infection, macroscopic assessment of the faeces with respect to diarrhea and excretion of blood on days 5 and 7 p.i. (assessment 0 to 6), macroscopic assessment of the intestinal mucosa, in particular of the appendices (assessment 0 to 6) and the oocyst excretion as well as the proportion (in %) of the oocysts sporulating in the course of 24 hours. The number of oocysts in the faeces was determined with the aid of a McMaster counting chamber (see Engelbrecht and coworkers “Parasitologische Arbeitsmethoden in Medizin und Veterinärmedizin” [Parasitological Working Methods in Medicine and Veterinary Medicine], Akademie-Verlag, Berlin (1965)). The individual findings are related to the untreated non-infected control groups and a total score is calculated (cf. A. Haberkorn (1986), pp. 263 to 270 in Research in Avian Coccidiosis ed. L. R. McDougald, L. P. Joyner, P. L. Long, Proceedings of the Georgia Coccidiosis Conference, Nov. 18-20, 1985, Athens/Georgia USA).

Experimental results with combinations according to the invention are shown by way of example in the following table. The synergistic activity of the combinations in comparison with the individual components is particularly evident in the reduction of oocyst excretion and with respect to the section findings.

In the following tables, in the column “Treatment” the information means

n.inf.contr.=non-infected control group

inf.contr.=infected control group

(I-A)=benzimidazole of the formula (I-A).

In the column “ppm”, the concentration of the active compound employed in the feed is indicated in ppm.

In the column “mortality”, the percentage of the dead animals is indicated under % and the number of dead animals/animals employed in the experiment is indicated under n.

In the column “weight % of not inf. control”, the ratio of the weight of the treated animals to the weight of the non-infected control group is indicated.

In the columns “dropping scores”, “lesion score” and “oocyst control”, individual details of the action are given.

In the column “% efficacy”, the total score is assessed; 0% means no action, 100% means full action.

TABLE

| | | | | Weight in | | | Oocysts in | |
|---|---|---|---|---|---|---|---|---|
| | | Mortality | | % of not | Dropping | Lesion | % of inf. | % |
| Treatment | ppm | % | n | inf. control | score | score | control | efficacy |
| Not infected | 0 | 0 | 0/6 | 100 | 0 | 0 | 0.3 | 100 |
| control | | | | | | | | |
| Infected | 0 | 33.3 | 2/6 | 30.5 | 6 | 6 | 100 | 0 |
| control | | | | | | | | |
| (I-A)* | 1 | 0 | 0/3 | 16 | 6 | 6 | 36.0 | 15.7 |
| (I-A)* | 2.5 | 0 | 0/3 | 41 | 6 | 6 | 80.7 | 12.3 |
| Diclazuril | 0.05 | 0 | 0/3 | 86 | 0 | 0 | 17.0 | 69.3 |
| Diclazuril | 0.1 | 0 | 0/3 | 92 | 0-2 | 0 | 14.0 | 77.0 |
| (I-A)* + diclazuril | 1 + 0.05 | 0 | 0/3 | 85 | 0 | 0 | 6.0 | 78.3 |
| (I-A)* + diclazuril | 1 + 0.1 | 0 | 0/3 | 71 | 0-1 | 0 | 5.7 | 67.3 |
| (I-A)* + diclazuril | 2.5 + 0.05 | 0 | 0/3 | 85 | 0 | 0 | 6.0 | 76.7 |
| (I-A) + diclazuril | 2.5 + 0.1 | 0 | 0/3 | >100 | 0 | 0 | 3.7 | 91 |
| (I-A)* | 1 + 1 | 0 | 0/3 | 95 | 0 | 0 | 0 | 100 |
| diclazuril | | | | | | | | |

*contains about 23% of the compound of the formula (I-C)

1. Products, comprising at least one substituted benzimidazole effective against parasitic protozoa and at least one 1,2,4-triazine derivative.

2. Products according to claim 1 for the simultaneous, separate or successive use against parasitic protozoa in humans or animals.

3. Products according to either of the preceding claims where the substituted benzimidazole effective against parasitic protozoa is a compound of the formula (I) or a salt thereof, if appropriate in the form of a hydrate or solvate, 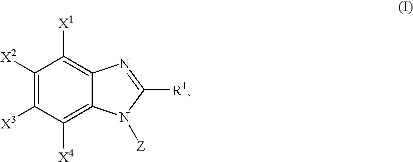
in which
Z represents hydrogen or the radical —CHR²R³,
R¹ represents fluoroalkyl,
R² represents hydrogen or alkyl,
R³ represents a radical of the formula 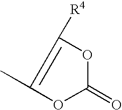
or represents a radical of the formula 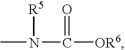
R⁴ represents alkyl,
R⁵ represents alkyl or substituted phenyl,
R⁶ represents alkyl,
X¹, X², X³ and X⁴ independently of one another represent hydrogen, halogen, halo-alkyl, haloalkoxy, haloalkylthio or haloalkylsulphonyl,
or else
X² and X³ or X³ and X⁴ together represent a dioxyhaloalkylene radical.

4. Products according to claim 3, characterized in that Z represents the radical —CHR²R³.

5. Products according to any of the preceding claims in which the 1,2,4-triazine derivative is a compound of the formula (II) or a salt thereof, if appropriate in the form of a hydrate or solvate, 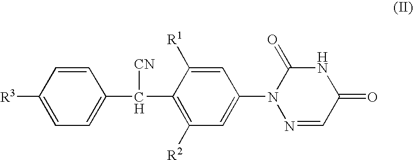
in which
R¹ and R² independently of one another represent hydrogen or Cl and
R³ represents fluorine or chlorine.

6. Products according to claim 4 in which the 1,2,4-triazine derivative is clazuril, letrazuril or diclazuril.

7. Use of at least one substituted benzimidazole effective against parasitic protozoa and at least one 1,2,4-triazine derivative for preparing products for controlling parasitic protozoa.

8. Use of a compound of the formula (I) 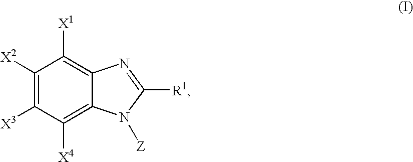
in which
Z represents hydrogen,
R¹ represents fluoroalkyl,
X¹, X², X³ and X⁴ independently of one another represent hydrogen, halogen, haloalkyl, haloalkoxy, haloalkylthio or haloalkylsulphonyl,
or else
X² and X³ or X³ and X⁴ together represent a dioxyhaloalkylene radical,
or a salt thereof, if appropriate in the form of a hydrate or solvate, for preparing products for controlling parasitic protozoa.

9. Method for controlling parasitic protozoa in humans or animals, where effective amounts of at least one substituted benzimidazole effective against parasitic protozoa and at least one 1,2,4-triazine derivative are administered to the human or animal.

10. Method for controlling parasitic protozoa in humans or animals, where at least one substituted benzimidazole, effective against parasitic protozoa, of the formula (I) 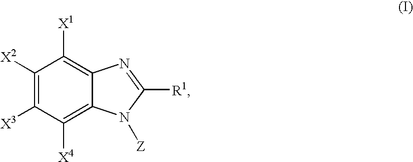
in which
Z represents hydrogen,
R¹ represents fluoroalkyl,
X¹, X², X³ and X⁴ independently of one another represent hydrogen, halogen, haloalkyl, haloalkoxy, haloalkylthio or haloalkylsulphonyl,
or else
X² and X³ or X³ and X⁴ together represent a dioxyhaloalkylene radical,
or a salt thereof, if appropriate in the form of a hydrate or solvate, is administered in an effective amount to the human or animal.
